(19) United States
(12) Patent Application Publication
(10) Pub. No.: US 2025/0120360 A1
(43) Pub. Date: Apr. 17, 2025

(54) CORYLUS PLANT NAMED 'THOMPSON'

(71) Applicant: Oregon State University, Corvallis, OR (US)

(72) Inventor: Shawn A. Mehlenbacher, Corvallis, OR (US)

(73) Assignee: Oregon State University, Corvallis, OR (US)

(21) Appl. No.: 18/484,961

(22) Filed: Oct. 11, 2023

(51) Int. Cl. A01H 6/00; A01H 5/02

(52) U.S. Cl. CPC A01H 6/00; A01H 5/02

(57) ABSTRACT

A new and distinct Corylus avellana plant is disclosed, distinguished by its low vigor, globose growth habit, production of nuts with round kernels that fall free of the husk at maturity, resistance to eastern filbert blight and big bud mite, S-alleles, nut size, kernel percentage, frequency of nut defects, time of pollen shed, and length of the husk or involucre.

### ACKNOWLEDGMENT OF GOVERNMENT SUPPORT

This invention was made with government support under Specialty Crops Research Initiative Grant 2016-51181-25412 awarded by the USDA-National Institute of Food and Agriculture and under agreement 58-5358-4-025 awarded by USDA-Agricultural Research Service. The government has certain rights in the invention.

### INCORPORATION OF ELECTRONIC SEQUENCE LISTING

The Sequence Listing is submitted as an XML file named “Sequence.xml,” created on Oct. 3, 2023, 35,809 bytes, which is incorporated by reference herein.

### BACKGROUND

The present invention relates to a new and distinct cultivar of European hazelnut, also known as filbert, and botanically known as Corylus avellana, hereinafter referred to by the name ‘Thompson’. The new Corylus plant resulted from a controlled cross of the female parent Corylus avellana ‘OSU 914.082’ (unpatented) x male parent Corylus avellana ‘OSU 820.007’ (unpatented) made in February 2005 (see, FIG. 1). A tree of ‘OSU 914.082’ was emasculated and covered to prevent foreign pollen contamination. Controlled pollinations used pollen of ‘OSU 820.007’. Hybrid seeds resulting from the cross were harvested in August 2005. They were provided a period of moist chilling, subsequently germinated, and the seedlings were grown in the greenhouse during the summer of 2006. From this cross, a total of 93 seedling trees were planted in a research field in Corvallis, OR, in October 2006. ‘Thompson’ was discovered and selected as a single plant within that progeny of the stated cross-pollination. It was originally assigned the designation ‘OSU 1304.039’, which indicates the row and tree location of the original seedling.

The female parent is ‘OSU 914.082’, obtained from a cross of ‘OSU 315.096’ (unpatented) and ‘Santiam’ (unpatented, Mehlenbacher et al., HortScience 42:715-717, 2007). ‘OSU 315.096’ is from a cross of ‘OSU 23.017’ (unpatented) and ‘Tonda Gentile delle Langhe’ (unpatented) from northern Italy. ‘OSU 23.017’ is from a cross of ‘Barcelona’ (unpatented) and ‘Extra Ghiaghli’ (unpatented). ‘Barcelona’ is an old Corylus avellana cultivar from Spain that is widely distributed in Europe and was introduced to the U.S. in about 1885 (Mehlenbacher and Miller, Fruit Var. J. 43:90-95, 1989). ‘Barcelona’ is known under several different names, including ‘Castanyera’ in Tarragona (Spain), ‘Grande’ in Asturias (Spain), ‘Grada de Viseu’ in Portugal, and ‘Fertile de Coutard’ in France. ‘Extra Ghiaghli’, described by Raptopolous and Kantartzis (1961), is a clone of the important Turkish cultivar ‘Tombul’ and was imported from Greece in the late 1950s. ‘Santiam’ is a parent of ‘McDonald’ (US PP28,200).

The male parent is ‘OSU 820.007’, an unreleased selection from a cross of ‘OSU 275.031’×‘OSU 504.065’ (both unpatented). ‘OSU 275.031’ is from a cross of ‘Montebello’ (unpatented) from Sicily and ‘OSU 74.037’ (unpatented). The parentage of ‘OSU 74.037’ includes ‘Barcelona’, ‘Daviana’ (unpatented) from England and ‘Tombul Ghiaghli’ (unpatented), a Turkish type from Greece (Raptopolous and Kantartzis, 1961). ‘OSU 504.064’ is a parent of ‘York’ (US PP24,972). The pedigree of ‘OSU 504.065’ includes ‘Montebello’, grower selection ‘Compton’ (unpatented), an unknown selection labeled F-4, and ‘Gasaway’ (unpatented, Mehlenbacher et al., HortScience 26:410-411, 1991), the original donor of resistance to eastern filbert blight.

‘Thompson’ was asexually reproduced by rooted suckers in 2012 through 2021 in Corvallis, OR. The unique features of this new Corylus are stable and reproduced true-to-type in successive generations of asexual reproduction.

### SUMMARY

The following traits have been observed and are determined to be the unique characteristics of ‘Thompson’. These characteristics in combination distinguish ‘Thompson’ from other know Corylus avellana cultivars, such as ‘Yamhill’ (unpatented, Mehlenbacher et al., HortScience 44:845-847, 2009), ‘Jefferson’ (unpatented, Mehlenbacher et al., HortScience 46:662-664, 2011), ‘McDonald’ (US PP28,200), ‘Wepster’ (US PP27,141), ‘Barcelona’, and ‘Tonda di Giffoni’ (unpatented), and other cultivars and selections of Corylus avellana known to the inventor:
1. Low vigor and globose plant habit.
2. High level of resistance to eastern filbert blight (EFB) caused by the fungus Anisogramma anomala (Peck) E. Müller. The source of this resistance is a single dominant allele conferred from ‘Gasaway’, which protects Corylus avellana ‘McDonald’, ‘Wepster’, ‘Dorris’ (US PP25,022), ‘Jefferson’, ‘Yamhill’, and several other Corylus avellana cultivars and pollinizers.
3. Expression of incompatibility alleles S₂ and S₁₅ in the styles and S₁₅ in the pollen.
4. High resistance to big bud mites (primarily Phytoptus avellanae Nal.).
5. Other distinguishing characteristics include nut size, kernel percentage (ratio of kernel weight to nut weight), frequency of defects (blank nuts, moldy kernels, twin kernels, etc.), time of pollen shed, and length of the husk or involucre.

Female Receptivity: The female inflorescences of ‘Thompson’ emerge early and are fully receptive in mid-January, with ‘McDonald’, but their styles blacken about two weeks earlier than those of ‘McDonald’ (see, FIGS. 5-7).

Pollen Shed: ‘Thompson’ generally sheds pollen in Corvallis, OR in early mid-season, with ‘McDonald’ and before ‘Felix’ (US PP24,973) (see, FIGS. 5-7).

Husk Length: The husk of ‘Thompson’ is about 1.3 times nut length, slightly shorter than ‘Barcelona’, while ‘Wepster’ is 2.0 times nut length. The husks have finger-like tips, are slit down the side, and flare open as they dry at maturity (see, FIG. 3). About 92% of the nuts fall free of the husk at maturity.

Nuts and Kernels: ‘Thompson’ produces round, medium-sized nuts and kernels. Most of the pellicle is removed from the kernel with dry heat in the blanching process (see, FIG. 4). The combination of correct size, round shape and good blanching make ‘Thompson’ well-suited to the kernel market for use in confections and baked goods.

The foregoing and other objects and features of the disclosure will become more apparent from the following detailed description, which proceeds with reference to the accompanying figures.

### BRIEF DESCRIPTION OF THE DRAWINGS

The accompanying color photographs illustrate the overall appearance of the new variety, showing the colors as true as it is reasonably possible to obtain in colored reproductions of this type. It should be noted that colors may vary, for example due to lighting conditions at the time the photograph is taken. Therefore, color characteristics of this new variety should be determined with reference to the observations described herein, rather than from the photographs alone.

FIG. 1 shows the pedigree of new cultivar ‘Thompson’ (OSU 1304.039).

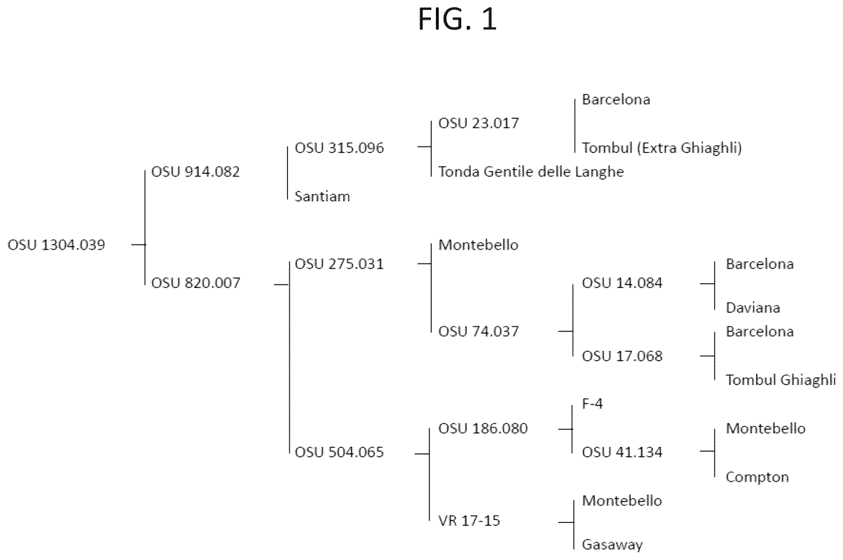

FIG. 2 shows a tree of the new hazelnut cultivar ‘Thompson’ on Apr. 6, 2022, in the eighth leaf pruned to a single trunk.

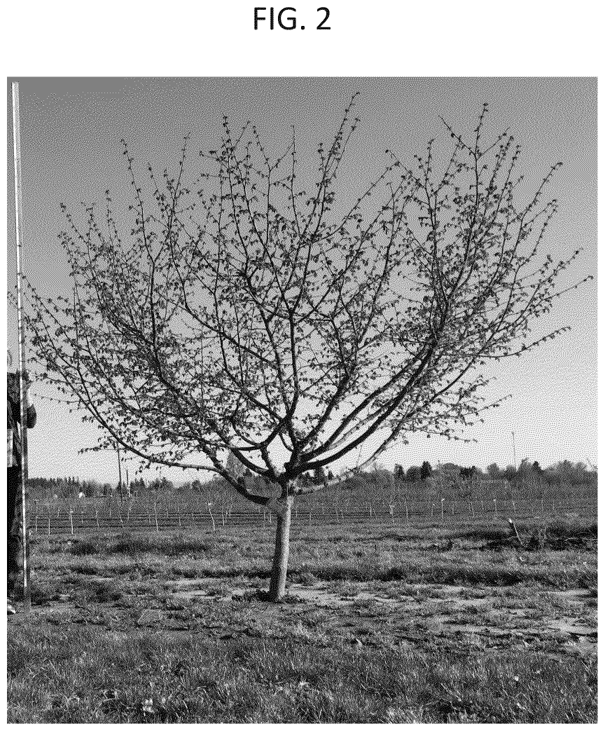

FIG. 3 shows immature nuts and husks of ‘Thompson’ and ‘McDonald’ hazelnuts.

FIG. 4 shows nuts, raw kernels, and blanched kernels of hazelnuts of ‘Thompson’(OSU 1304.039) and ‘McDonald’ hazelnuts.

FIG. 5 shows the time of female receptivity (bottom, red), pollen shed (top, green), and vegetative budbreak of ‘Thompson’ (OSU 1304.039), ‘McDonald’ and other hazelnut cultivars in Corvallis, OR (December 2018 to March 2019).

FIG. 6 shows the time of female receptivity (bottom, red), pollen shed (top, green), and vegetative budbreak of ‘Thompson’ (OSU 1304.039), ‘McDonald’ and other hazelnut cultivars in Corvallis, OR (December 2019 to March 2020).

FIG. 7 shows the time of female receptivity (bottom, red), pollen shed (top, green), and vegetative budbreak of ‘Thompson’ (OSU 1304.039), ‘McDonald’ and other hazelnut cultivars in Corvallis, OR (December 2020 to March 2021).

### SEQUENCE LISTING

The nucleic acid sequences in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. In the accompanying sequence listing SEQ ID NOS: 1-40 are primer sequences that can be used for genetic fingerprinting (see, Table 7).

### DETAILED DESCRIPTION

Certain characteristics of this variety may change with changing environmental conditions (such as photoperiod, temperature, moisture, soil conditions, nutrient availability, or other factors) without, however, any variance in genotype. Color descriptions and other terminology are used in accordance with their ordinary dictionary descriptions unless the context clearly indicates otherwise. The aforementioned photographs and following observations and measurements describe plants grown in Corvallis, OR, outdoors in the field during the spring and summer. The plant used for the photographs and description were from a seven-year-old tree propagated by tie-off layerage and growing on its own roots. In the following description, color references are made to The Royal Horticultural Society Colour Chart, 5^{th} Edition, 2007 except where general terms of ordinary dictionary significance are used. The color values were determined in spring and summer 2020-2022 under natural light conditions in Corvallis, Oregon. The UPOV descriptor list is from the Mar. 28, 1979 Hazelnut guidelines from UPOV.

### Botanical

Scientific Name: Corylus avellana

### Parentage:

Seed Parent: ‘OSU 914.082’ (unpatented breeding selection)
Pollen Parent: ‘OSU 820.007’ (unpatented breeding selection)

### Incompatibility Alleles:

‘Thompson’ has incompatibility alleles S₂ and S₁₅. Hazelnut is a wind-pollinated, monoecious species that exhibits a sporophytic self-incompatibility system controlled by a single locus designated as the S-locus with 33 alleles (Mehlenbacher, J. Amer. Soc. Hort. Sci. 139:191-212, 2014). For comparison, Corylus avellana ‘McDonald’ has the same pair of alleles S₂ and S₁₅, ‘PollyO’ (US PP32,459) has alleles S₂ and S₁₀, ‘Sacajawea’ (unpatented, Mehlenbacher et al., HortScience 43:255 257, 2008) has the alleles S₁ and S₂₂, ‘Tonda di Giffoni’ has alleles S₂ and S₂₃, while ‘Tonda Pacifica’ (US PP22,715) and ‘Wepster’ have alleles S₁ and S₂.

### Propagation (Type Rooted Suckers):

Time to Initiate Roots: About 30 days at 20° C.
Time to Produce a Rooted Young Plant: About six months at 22° C.
Root Description: Fine to thick; freely branching; creamy white in color

### Propagation (Type Whip Grafting):

Time to Budbreak on the Scions: About 14 days at 25° C.
Time to Produce a Grafted Plant: About six months at 25° C.

### Plant:

Appearance: Natural habit is perennial shrub, but in commercial orchards, is a single trunk tree. Globose plant habit.
Growth and Branching Habit: Freely branching; about 15 lateral branches develop per plant. Pinching, that is, removal of the terminal apices, enhances branching with lateral branches potentially forming at every node.
Vigor: Low
Plant Height: about 2.9 m
Plant Diameter: about 3.1 m
Trunk Cross Section Area 30 cm Above the Soil Line: In a trial planted in Corvallis, Oregon in 2014, trunk cross-sectional area was 46.9 cm² in December 2020. In a second trial planted in Corvallis, OR in 2015, trunk cross-sectional area was 47.0 cm². These values are about 65% of ‘Jefferson’ in the same trials.
Trunk Color: RHS 197B

### Lateral Branch:

Length: 19.0-31.0 cm, typically 27.2 cm
Diameter: 3.3-4.5 mm, typically 3.7 mm
Internode Length (at base): About 0.75 cm
Internode Length (at tip): 2.4-3.5 cm, typically about 2.8 cm.
Texture: Smooth, pubescent
Strength: Strong
Color:
   Immature: RHS 144A
   Mature: RHS 177B
   Previous Seasons Branches: RHS 199C

### Foliage:

Arrangement: Alternate, simple
Size:
   Length: 7.5-11.9 cm, typically about 9.7 cm
   Width: 7.4-11.1 cm, typically about 8.8 cm
Shape: Oblong to ovate
Apex: Obtuse to acute
Base: Cordate
Margin: Serrate
Leaf Texture: Slightly pubescent on upper and lower surfaces
Foliage Color:
   Developing Foliage:
      Upper Surface: RHS 141B
      Lower Surface: RHS 141C
   Fully Expanded Foliage:
      Upper Surface: spring and summer, RHS 137B; late summer and fall, RHS 137B
      Lower Surface: spring and summer, RHS 137D; late summer and fall, RHS 137D
Venation Pattern: Pinnate
Venation Color:
   Upper Surface: Spring and summer, RHS 145A; late summer and fall, RHS 145A
   Lower Surface: Spring and summer, RHS 145A; late summer and fall, RHS 145A
Leaf Bud:
   Shape: Globular, Descriptor-2
   Time of Budbreak: Medium, Descriptor-5. ‘Thompson’ budbreak is about 12 days before ‘Jefferson’, 6 days before ‘Felix’, and 6 days later than ‘McDonald’.
   Color of leaf buds: RHS 144C
Petiole:
   Length: 13.0-19.3 mm, typically about 16.3 mm
   Diameter: 0.8-1.8 mm, typically about 1.3 mm
   Texture: Pubescent (both upper and lower surfaces)
   Color: RHS 143B (lower surface, early fall)

### Flowers:

Male Inflorescences:
   Catkins Color Prior to Elongation: RHS 176B exposed to sun, RHS 194C in shade.
   Catkin Length: 23.5 mm.
Female Inflorescence Style Color: RHS 059A
Time of Female Flowering: Early-medium, Descriptor-4
Time of Female Flowering Compared to Male Flowering: Homogamy, Descriptor-2
Involucre Constriction: Present
Involucre Length: 1.3 times length of nut, Descriptor-5
Size of Indentation: Strong, Descriptor-7
Strength of Serration of Indentation: Weak, Descriptor-3
Thickness of Callus at Base: Thin, Descriptor-3
Pubescence on Husk: Absent, Descriptor-1
Density of Hairiness of Involucre: Weak, Descriptor-3
Jointing of Bracts: Absent, Descriptor-1

### Nut:

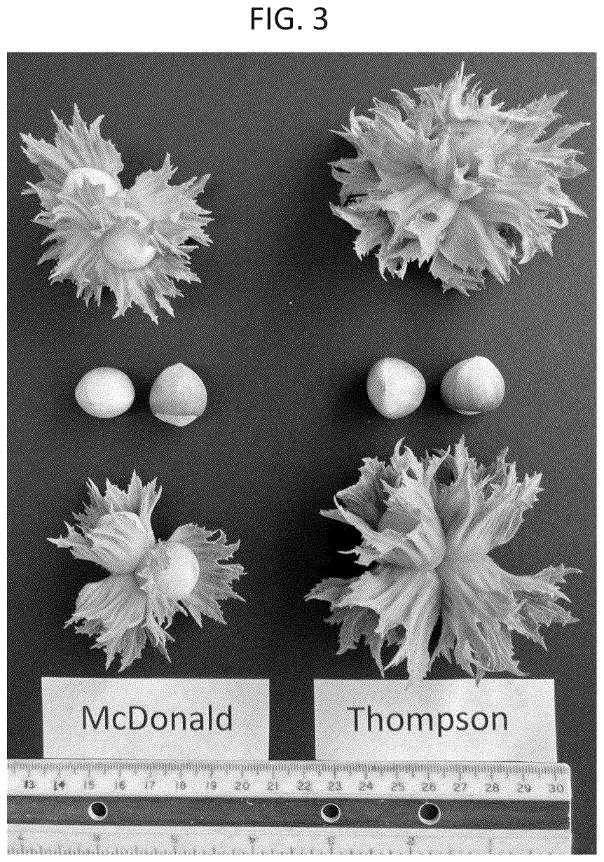

Length: Average 17.1 mm
Width: Average 18.6 mm
Depth: Average 16.8 mm
Nut Shape: Globular, Descriptor-1
Nut Shape Index: 1.04 ((Width+Depth)/2*Length)
Nut Compression Index: 1.10 (Width/Depth)
Nut Weight: 2.79 g
Kernel Weight: 1.25 g
Kernel Percentage: 44.8% (Kernel Weight/Nut Weight)
Number of Fruits per Cluster: Three to four (see, FIG. 3)
Nutshell Coloration: RHS 164A
Number of Stripes on Shell: Many, Descriptor-7
Shape of Fruit Apex: Flat, Descriptor-4
Prominence of Fruit Apex: Slightly prominent, Descriptor-3
Size of Fruit Pistil Scar on Shell: Small, Descriptor-3
Hairiness of Top of Fruit: Weak, Descriptor-3
Curvature of Nut Basal Scar: Flat, Descriptor-2
Double Kernels: Absent
Kernel Shape: Globular, Descriptor-1
Shape of Kernel in Cross-Section: Circular, Descriptor-2
Lateral Groove in Kernel: Absent, Descriptor-1
Corkiness of Pellicle of Kernel: Slightly corky, Descriptor-3

### Disease/Pest Resistance:

In Oregon, plants of ‘Thompson’ are resistant to EFB caused by the fungus Anisogramma anomala (Peck) E. Müller. All trees of ‘Thompson’ in trial plots have remained free of EFB with no fungicide applications. Fungicide applications are not expected to be needed to control EFB. Plants have not been challenged against all populations of Anisogramma anomala present in North America (Muehlbauer et al., Phytopathology 109:1074-1082, 2019); it is likely that ‘Thompson’ is susceptible to populations of the fungus in New Jersey that are able to overcome resistance derived from ‘Gasaway’.

Susceptibility to bacterial blight caused by Xanthomonas arboricola pv. corylina has not been quantified, but no trees in the trials in Corvallis, Oregon were affected.

Susceptibility to bud mite (primarily Phytoptus avellanae Nal.) was rated in trials in Corvallis, OR, in mid-December on a scale of 1 (no blasted buds) to 5 (many blasted buds). The average rating for ‘Thompson’ was 1.04, indicating a high level of resistance comparable to ‘Barcelona’ and ‘Jefferson’.

### Temperature Tolerance:

‘Thompson’ was selected in Corvallis, OR and is targeted for production in USDA Plant Hardiness Zones 6a to 7b. Plants of the new Corylus avellana have been observed to tolerate temperatures from −18° C. to 40° C.

### Comparative Data:

Disease Resistance: Based on field exposure and DNA markers, ‘Thompson’ is resistant to eastern filbert blight (EFB) caused by Anisogramma anomala in Oregon, where cultivars previously widely grown (‘Barcelona’, ‘Ennis’ (unpatented), ‘Daviana’, and ‘Butler’ (unpatented)) are susceptible. The more recently developed cultivars ‘Santiam’, ‘Yamhill’, ‘Jefferson’, ‘Dorris’, ‘Wepster’, and ‘McDonald’ and their associated pollinizers are protected from EFB by a single resistance gene from Corylus avellana ‘Gasaway’. This gene provides a high level of resistance in Oregon and Washington where the diversity of the fungus is limited (Muehlbauer et al., Phytopathology 109:1074-1082, 2019), but does not provide a similar level of protection from disease in the eastern U.S. where the pathogen is endemic and genetically diverse (Capik and Molnar, J. Amer. Soc. Hort. Sci. 137:157-172, 2012; Molnar et al., Plant Dis. 10:1265, 2010; Muehlbauer et al., Phytopathology 109:1074-1082, 2019). It is unlikely that ‘Thompson’ would remain free of EFB in New Jersey where populations of the pathogen are genetically diverse.

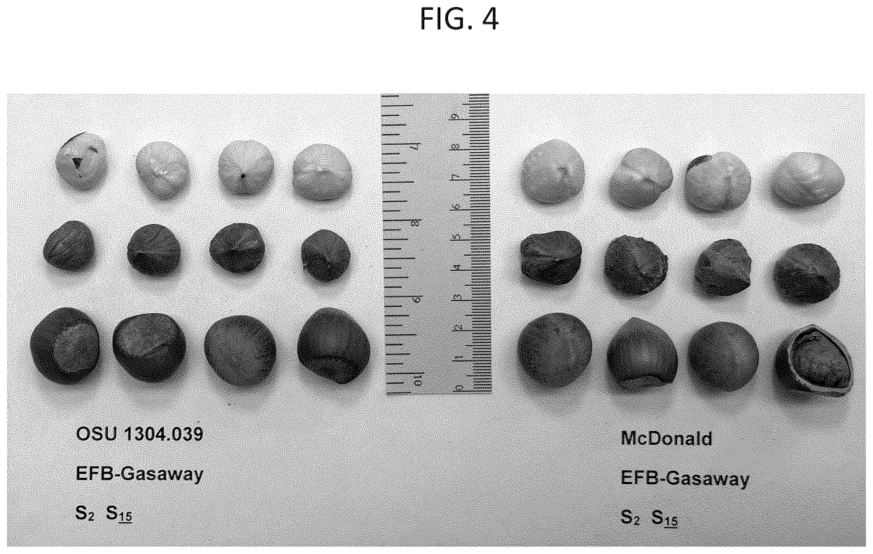

Nut and Kernel Characteristics: ‘Thompson’ hazelnut is targeted for the blanched kernel market and specifically for nut production in the Pacific Northwestern United States in USDA Plant Hardiness Zones 6a to 7b. As shown in FIGS. 3 and 4, the nut shape and kernel shape are round. The average single nut weight for ‘Thompson’ over four years (2017-20) is 2.79 g, average single kernel weight is 1.25 g, with an average kernel percentage of 44.8% (see, Table 3). Nut weight in the same trial was 3.86 g for ‘Jefferson’, 2.70 g for ‘McDonald’ and 2.43 g for ‘Wepster’. Kernel weights in the same trial were 1.74 g for ‘Jefferson’, 1.41 g for ‘McDonald’ and 1.13 g for ‘Wepster’. Kernel percentage in the same trial was 45.0% for ‘Jefferson’, 52.2% for ‘McDonald’ and 46.7% for ‘Wepster’. ‘Thompson’ nuts and kernels are significantly smaller than those of ‘Barcelona’ and ‘Jefferson’, and intermediate between ‘McDonald’ and ‘Wepster’. In the trial planted in 2014 in Corvallis, OR, the trees produced a few nuts in 2016, but were not harvested. Nuts were harvested for four years (2017-20), dried, weighed and evaluated. Total nut weight per tree (2017-20) was 14.7 kg for ‘Thompson’, compared to the checks ‘Jefferson’ (18.3 kg), ‘McDonald’ (18.6 kg) and ‘Wepster’ (21.8 kg). Trunk cross-sectional area (TCA) was 46.89 cm², or 66% of ‘Jefferson’. Yield efficiency, the ratio of total yield to TCA, was a very high 0.315 kg/cm² and higher than all three checks. Trees have a globose growth habit, and lower vigor than all other selections in the Corvallis trial.

Raw kernels of ‘Thompson’ have a light brown pellicle with little attached fiber (average rating was 2.19 on a scale of 1 (no fiber) to 4 (much fiber); see, Table 3). Pellicle removal after roasting at 150° C. for 15 min and rubbing is rated on a scale of 1 (complete pellicle removal) to 7 (no pellicle removal). Most of the pellicle on ‘Thompson’ kernels is removed after roasting with an average rating of 2.75 (see, Table 3), a value slightly better than ‘McDonald’ (3.50) and ‘Wepster’ (3.03).

The average percentage of good kernels (kernels free of defects) for ‘Thompson’ in the trial planted in 2014 was 83.2% (see, Table 3). There was on average 4.2% blank nuts, 2.8% moldy kernels, 1.6% nuts with shriveled kernels, and 7.4% poorly filled nuts. The percentage of good kernels for ‘Thompson’ was considerably higher than the 60-70% range in multiple reports for ‘Barcelona’ in Oregon. The percentage of poorly filled nuts for ‘Thompson’ (7.4%) was lower than for ‘Jefferson’ (12.6%) and ‘Wepster’ (17.3%) despite the heavy crop loads on ‘Thompson’ trees.

Nut Maturity Date: The nuts of ‘Thompson’ are typically borne in clusters of 3-4 in husks about 30% longer than the nuts (see, FIG. 3). The husks open as they dry at maturity. About 92% of the nuts fall free of the husk at maturity and the other 8% of the nuts come out of the husks as they moved through the harvester. When mature, the shells are tan in color. Estimates recorded at the time of harvest in the two trials indicate that ‘Thompson’ nuts mature about 3 days before ‘Barcelona’, 6 days before ‘Jefferson’, 7 days after ‘Wepster’, and 10 days after ‘McDonald’.

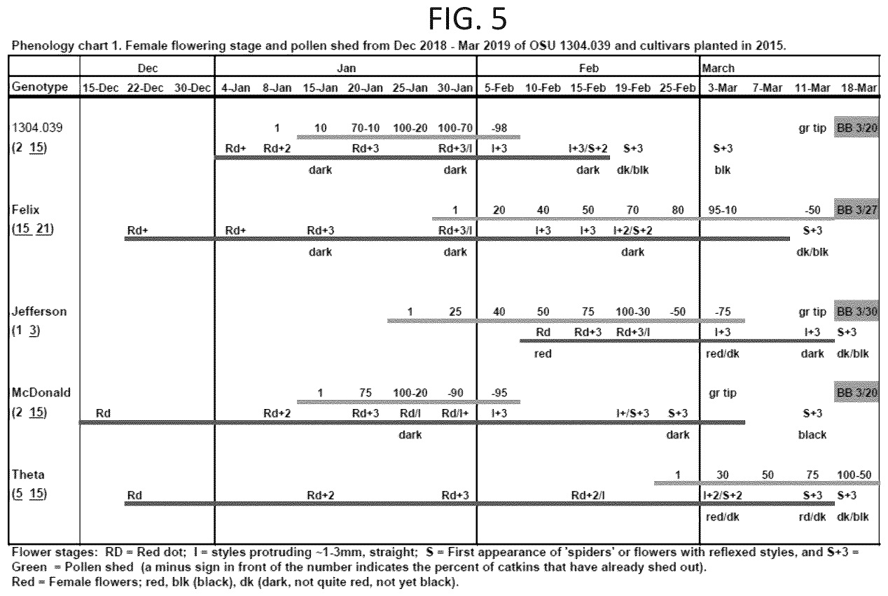

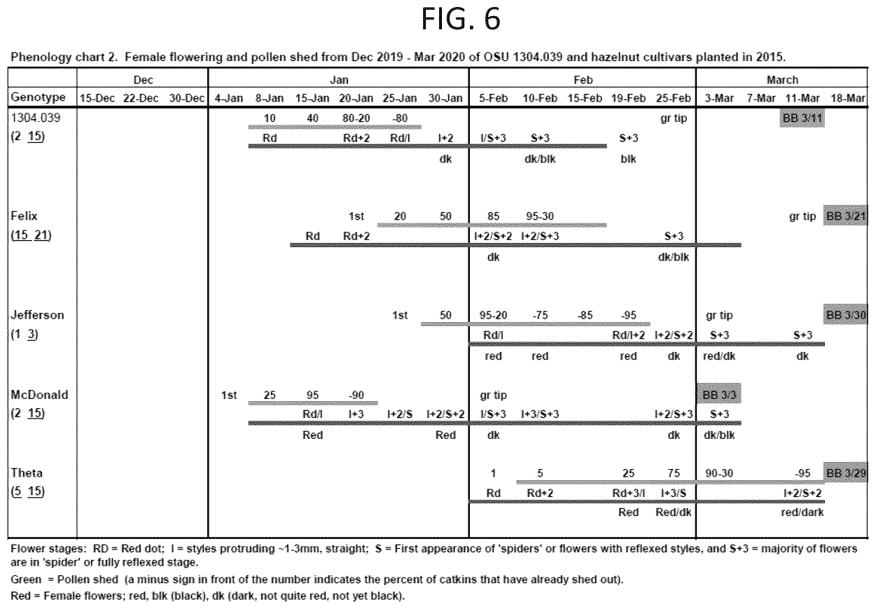

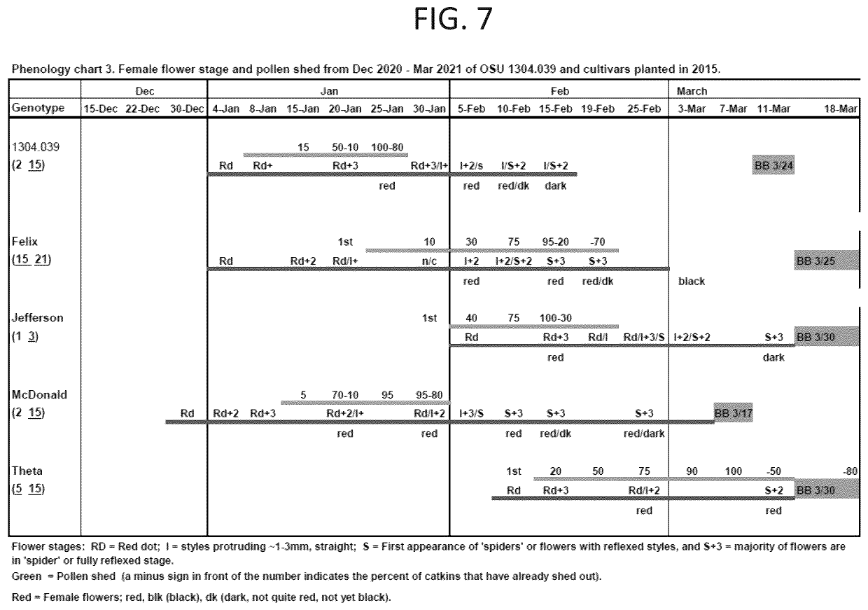

Flowering Time: Trees of ‘Thompson’ set a high number of catkins that shed copious amounts of pollen. Time of pollen shed and female receptivity were recorded weekly from mid-December to mid-March in the second trial for three years for ‘Thompson’ and the check cultivars (see, FIGS. 5-7). The female inflorescences of ‘Thompson’ emerge early and are fully receptive in mid-January, with ‘McDonald’, but their styles blacken about two weeks earlier than those of ‘McDonald’. Female receptivity dates are similar for ‘Wepster’, ‘Yamhill’, ‘York’ and ‘PollyO’. Pollen is shed by ‘Thompson’ in early mid-season, with ‘McDonald’ and before ‘Felix’. Pollen has been collected and used in several controlled pollinations, and both quantity and viability appear to be very good. Pollen germination tests over three years (2018-2020) showed good germination for ‘Thompson’. On Jan. 23, 2020, pollen germination of ‘Thompson’ was 79% in contrast to 40% for ‘Barcelona’ while values for other cultivars ranged from 69 to 91%. On Jan. 30, 2019, pollen germination of ‘Thompson’ was 76% and similar to ‘PollyO’ (72%), ‘Sacajawea’ (82%) and ‘Yamhill’ (72%). Pollen germination tests on five dates in January 2018 showed good germination for ‘Thompson’ (65%), ‘PollyO’ (77%), ‘Sacajawea’ (73%) and ‘Yamhill’ (81%), and much lower germination for ‘Barcelona’ (40%). When establishing a new orchard, the inclusion of at least three pollinizers with the main cultivar is encouraged.

Additional Comparative Descriptors: Tables 1-6 provide additional descriptors and trial data distinguishing ‘Thompson’ from various hazelnut varieties.

Microsatellite Marker Analysis: Twenty microsatellite (simple sequence repeat) markers were used. PCR products were multiplexed post-PCR and sized using capillary electrophoresis. See, for example, Bassil et al., Acta Horticulturae 686:105-110, 2005; Gökirmak et al., Genetic Resources and Crop Evolution 56:147-172, 2009; Gürcan and Mehlenbacher. Molecular Breeding 26:551-559, 2010; Gürcan et al. Tree Genetics and Genomes 6:513-531, 2010). The microsatellite markers are described in Table 7 and allele sizes are reported in Table 8.

TABLE 1
Nut yield, trunk cross-sectional area (TCA), yield efficiency, and bud mite ratings
of ‘Thompson’ in comparison with other hazelnut cultivars and selections
in the first trial, planted in 2014. Randomized complete block design with four blocks and
a single tree of each selection in each block. Yield is in kg per tree. Trunk cross-sectional
area (TCA) is in cm². Yield efficiency is in kg/cm². Bud mite ratings are from
1 (no blasted buds) to 5 (many blasted buds).

| | Nut Yield (kg) | | | | | | Yield | |
|---|---|---|---|---|---|---|---|---|
| Selection | 2017 | 2018 | 2019 | 2020 | Total | TCA | efficiency | BBM |
| ‘541.147’ | 0.367 | 2.875 | 4.758 | 3.670 | 11.669 | 71.13 | 0.1647 | 3.4 |
| ‘Thompson’ | 1.784 | 2.945 | 3.623 | 6.353 | 14.704 | 46.89 | 0.3151 | 1.0 |
| ‘Jefferson’ | 2.389 | 4.765 | 4.596 | 6.585 | 18.335 | 71.22 | 0.2581 | 1.2 |
| ‘McDonald’ | 0.782 | 3.895 | 5.858 | 8.323 | 18.857 | 79.59 | 0.2386 | 2.0 |
| ‘Wepster’ | 2.161 | 4.590 | 5.325 | 9.695 | 21.771 | 77.33 | 0.2867 | 1.5 |
| LSD (0.05) | 0.581 | 0.948 | 1.114 | 1.395 | 2.551 | 13.34 | 0.0448 | 0.2 |

TABLE 2
Nut weight, kernel weight, percent kernel, and ratings for fiber
and blanching (pellicle removal) of ‘Thompson’ in comparison
with other hazelnut cultivars and selections in the first trial,
planted in 2014. Randomized complete block design with four blocks
and a single tree of each selection in each block.

| Selection | 10-NutWt | 10-KerWt | Percent kernel | Fiber | Blanch |
|---|---|---|---|---|---|
| ‘541.147’ | 26.46 | 11.72 | 44.32 | 3.78 | 4.50 |
| ‘Thompson’ | 27.88 | 12.48 | 44.77 | 2.19 | 2.75 |
| ‘Jefferson’ | 38.64 | 17.37 | 44.99 | 2.84 | 4.06 |
| ‘McDonald’ | 26.96 | 14.05 | 52.15 | 2.59 | 3.50 |
| ‘Wepster’ | 24.27 | 11.34 | 46.73 | 2.75 | 3.03 |
| LSD (0.05) | 0.70 | 0.34 | 0.52 | 0.20 | 0.24 |

TABLE 3
Frequency of good nuts and of nut and kernel defects in ‘Thompson’ and other hazelnut
cultivars from the first replicated trial, planted in 2014, with 4 trees per selection.
Percentages of good nuts and nut kernel defects are averaged over four years (2017-2020).

| Selection | Good | Blank | Brown Stain | Moldy | Shrivel | Poor Fill | Twin | Black Tip |
|---|---|---|---|---|---|---|---|---|
| ‘541.147’ | 86.76 | 8.26 | 0.18 | 0.32 | 1.88 | 2.50 | 0.12 | 0.00 |
| ‘Thompson’ | 83.18 | 4.18 | 0.56 | 2.82 | 1.56 | 7.38 | 0.18 | 0.38 |
| ‘Jefferson’ | 77.76 | 3.82 | 1.62 | 2.06 | 1.44 | 12.62 | 0.62 | 1.00 |
| ‘McDonald’ | 84.38 | 3.26 | 0.44 | 2.00 | 4.62 | 5.00 | 0.06 | 0.26 |
| ‘Wepster’ | 73.82 | 6.00 | 0.12 | 1.00 | 0.76 | 17.26 | 0.06 | 1.06 |
| LSD (0.05) | 3.37 | 1.63 | 0.85 | 1.09 | 1.31 | 2.35 | 0.52 | 0.66 |

TABLE 4
Performance of hazelnut selections from the second replicated trial planted in 2015.
Trunk cross-sectional area (TCA), annual and cumulative field-run nut yield, and
yield efficiency of selections over four years (2018-2021). 7 trees per cultivar.

| | | | | | | Total field-run | | Yield efficiency | |
|---|---|---|---|---|---|---|---|---|---|
| | TCA | Field-run nut yield (kg/tree) | | | | yield (kg/tree) | | (total kg/cm²) | |
| Selection^{z} | (cm²) | 2018 | 2019 | 2020 | 2021 | Nut | Kernel | Nut | Kernel |
| ‘Thompson’ | 47 | 1.76 | 2.71 | 3.54 | 6.09 | 14.10 | 6.20 | 0.30 | 0.13 |
| ‘Felix’ | 122 | 1.00 | 2.81 | 3.93 | 6.96 | 14.70 | 7.47 | 0.12 | 0.06 |
| ‘Jefferson’ | 74 | 3.00 | 4.66 | 4.51 | 8.93 | 21.11 | 9.38 | 0.29 | 0.13 |
| ‘McDonald’ | 81 | 1.20 | 4.10 | 6.00 | 8.43 | 19.73 | 10.26 | 0.24 | 0.13 |
| ‘Theta’ | 123 | 0.57 | 2.11 | 0.59 | 3.64 | 6.91 | 3.52 | 0.06 | 0.03 |
| LSD _{(.05)} | 16 | 0.43 | 0.82 | 0.92 | 1.06 | 2.33 | 1.07 | 0.03 | 0.02 |

TABLE 5
Nut weight, kernel weight, kernel percentage, and rating for blanching
(pellicle removal) of ‘Thompson’ and 4 other hazelnut cultivars
in the second replicated trial planted in 2015. 7 trees per cultivar.

| | Nut | Kernel | | |
|---|---|---|---|---|
| Selection^{z} | weight (g) | weight (g) | Percent kernel | Blanch |
| ‘Thompson’ | 2.5 | 1.1 | 44 | 2.4 |
| ‘Felix’ | 2.5 | 1.2 | 51 | 2.2 |
| ‘Jefferson’ | 3.4 | 1.5 | 44 | 4.6 |
| ‘McDonald’ | 2.4 | 1.2 | 52 | 3.2 |
| ‘Theta’ | 2.1 | 1.0 | 50 | 2.6 |
| LSD_{(.05)} | 0.15 | 0.1 | 1.5 | 0.3 |

TABLE 6
Frequency of good nuts, and of nut and kernel defects of ‘Thompson’ in comparison
with other hazelnut cultivars and selections in the second trial, planted in 2015.

| | Frequency (%) | | | | | | | | | |
|---|---|---|---|---|---|---|---|---|---|---|
| | | | Brown | | Usable | | Usable | | | |
| Selection^{z} | Good | Blanks | Stain | Mold | Shrivel | Shrivel | Poor Fill | Poor Fill | Twin | Black Tip |
| ‘Thompson’ | 96.0 | 2.0 | 0.1 | 0.8 | 0.7 | 0.7 | 1.6 | 0.2 | 0.04 | 0.2 |
| ‘Felix’ | 95.0 | 4.0 | 0.0 | 0.1 | 0.7 | 0.8 | 0.7 | 0.3 | 0.2 | 0.1 |
| ‘Jefferson’ | 96.0 | 3.1 | 0.0 | 0.5 | 0.8 | 0.2 | 0.6 | 0.1 | 0.5 | 0.1 |
| ‘McDonald’ | 97.0 | 2.1 | 0.1 | 0.5 | 2.3 | 0.5 | 1.0 | 0.1 | 0.0 | 0.1 |
| ‘Theta’ | 95.0 | 4.6 | 0.0 | 0.1 | 0.2 | 0.1 | 0.7 | 0.1 | 0.1 | 0.04 |
| LSD _{(.05)} | 2.0 | 2.8 | 0.2 | 0.9 | 1.3 | 0.8 | 3.9 | 1.2 | 0.7 | 0.4 |

TABLE 7
Primers and annealing temperatures for the microsatellite marker loci used for
fingerprinting hazelnut cultivars. Allele size = range of sizes; Primers, forward
(F, listed first; 5′-3′) and reverse (R, second, 5′-3′) (HEX and FAM are dyes);
Tm = Annealing temperature ; n = Number of alleles; He = expected heterozygosity;
Ho = observed heterozygosity; PIC = Polymorphism information content;
r = frequency of null alleles; LG = linkage group (S is susceptible female parent
‘OSU 252.146’; R is resistant parent ‘OSU 414.062’); Reference is the journal
article where additional details were published (Gürcan et al., Tree Genetics
and Genomes 6: 513-531, 2010; Gurcan and Mehlenbacher, Molecular
Breeding 26: 551-559, 2010; Bassil, et al., Acta Horticulturae 686: 105-110,
2005; and Gökirmak, et al. Genetic Resources and Crop Evolution 56: 147-172, 2009).

| | Allele | Primer | Primer | Tm | | | | | | | |
|---|---|---|---|---|---|---|---|---|---|---|---|
| Locus | sizes | (SEQ ID NO) | (SEQ ID NO) | (° C.) | n | He | Ho | PIC | r | LG | Reference |
| A613 | 149- | Ned- | R- | 60 | 14 | 0.86 | 0.85 | 0.85 | 0.00 | 11R | Gurcan et al., |
| | 177 | CACACGCCTTGTC | CCCCTTTCACATG | | | | | | | | 2010 |
| | | ACTCTTT (1) | TTTGCTT (2) | | | | | | | | |
| A614 | 125- | Hex- | R- | 60 | 14 | 0.85 | 0.85 | 0.84 | 0.00 | 6S, | Gurcan et al., |
| | 156 | TGGCAGAGCTTTG | GCAGTGGAGGAT | | | | | | | 6R | 2010 |
| | | TCAGCTT (3) | TGCTGACT (4) | | | | | | | | |
| A616 | 136- | Fam- | R- | 60 | 13 | 0.85 | 0.85 | 0.83 | 0.00 | 8R | Gurcan et al., |
| | 162 | CACTCATACCGCA | ATGGCTTTTGCTT | | | | | | | | 2010 |
| | | AACTCCA (5) | CGTTTTG (6) | | | | | | | | |
| A640 | 354- | F- | Fam- | 67 | 11 | 0.80 | 0.73 | 0.77 | 0.04 | 10R | Gurcan et al., |
| | 378 | TGCCTCTGCAGTT | CGCCATATAATTG | | | | | | | | 2010 |
| | | AGTCATCAAATGT | GGATGCTTGTTG | | | | | | | | |
| | | AGG (7) | (8) | | | | | | | | |
| B617 | 280- | Fam- | R- | 60 | 9 | 0.80 | 0.78 | 0.78 | 0.01 | 8S, | Gurcan et al., |
| | 298 | TCCGTGTTGAGTA | TGTTTTTGGTGGA | | | | | | | 8R | 2010 |
| | | TGGACGA (9) | GCGATG (10) | | | | | | | | |
| B619 | 146- | Fam- | R- | 60 | 14 | 0.88 | 0.88 | 0.87 | 0.00 | 3S, | Gurcan et al., |
| | 180 | AGTCGGCTCCCCT | GCGATCTGACCTC | | | | | | | 3R | 2010 |
| | | TTTCTC (11) | ATTTTTG (12) | | | | | | | | |
| B634 | 218- | Hex- | R- | 60 | 9 | 0.76 | 0.76 | 0.73 | 0.00 | 4R | Gurcan et al., |
| | 238 | CCTGCATCCAGG | GTGCAGAGGTTG | | | | | | | | 2010 |
| | | ACTCATTA (13) | CACTCAAA (14) | | | | | | | | |
| B657 | 210- | Ned- | R- | 60 | 8 | 0.84 | 0.98 | 0.82 | −0.08 | 11S, | Gurcan et al., |
| | 228 | GAGAGTGCGTCTT | AGCCTCACCTCCA | | | | | | | 11R | 2010 |
| | | CCTCTGG (15) | ACGAAC (16) | | | | | | | | |
| B671 | 221- | Hex- | R- | 60 | 13 | 0.86 | 0.88 | 0.84 | −0.01 | 9S, | Gurcan et al., |
| | 249 | TTGCCAGTGCATA | ACCAGCTCTGGG | | | | | | | 9R | 2010 |
| | | CTCTGATG (17) | CTTAACAC (18) | | | | | | | | |
| B709 | 219- | Ned- | R- | 60 | 8 | 0.74 | 0.76 | 0.70 | −0.01 | 5S, | Gurcan et al., |
| | 233 | CCAAGCACGAAT | GCGGGTTCTCGTT | | | | | | | 5R | 2010 |
| | | GAACTCAA (19) | GTACACT (20) | | | | | | | | |
| B733 | 161- | Ned- | R- | 60 | 8 | 0.68 | 0.68 | 0.63 | 0.00 | 7S, | Gurcan et al., |
| | 183 | CACCCTCTTCACC | CATCCCCTGTTGG | | | | | | | 2R | 2010 |
| | | ACCTCAT (21) | AGTTTTC (22) | | | | | | | | |
| B749 | 200- | Hex- | R- | 60 | 6 | 0.60 | 0.64 | 0.51 | −0.03 | 1R | Gurcan et al., |
| | 210 | GGCTGACAACAC | TCGGCTAGGGTTA | | | | | | | | 2010 |
| | | AGCAGAAA (23) | GGGTTTT (24) | | | | | | | | |
| B751 | 141- | Fam- | R- | 60 | 7 | 0.80 | 0.78 | 0.77 | 0.01 | 7S, | Gurcan et al., |
| | 153 | AGCTGGTTCTTCG | AAACTCAAATAA | | | | | | | 2R | 2010 |
| | | ACATTCC (25) | AACCCCTGCTC | | | | | | | | |
| | | | (26) | | | | | | | | |
| B774 | 195- | Ned- | R- | 60 | 8 | 0.80 | 0.80 | 0.77 | 0.00 | 5S, | Gurcan et al., |
| | 213 | GTTTTGCGAGCTC | TGTGTGTGGTCTG | | | | | | | 5R | 2010 |
| | | ATTGTCA (27) | TAGGCACT (28) | | | | | | | | |
| C115 | 167- | Fam- | R- | 60 | 10 | 0.84 | 0.90 | 0.82 | −0.035 | 4S, | Bassil et al., |
| | 225 | CATTTTCCGCAGA | GTTTCCAGATCTG | | | | | | | 4R | 2005b; |
| | | TAATACAGG (29) | CCTCCATATAAT | | | | | | | | Gokirmak et |
| | | | (30) | | | | | | | | al., 2009 |
| KG807 | 226- | F- | Fam- | 54 | 4 | 0.67 | 0.78 | 0.60 | −0.07 | 11 | Gurcan and |
| | 248 | AAGCAAGAAAGG | CTTACAGATAAAT | | | | | | | | Mehlenbacher, |
| | | GATGGT (31) | GGCTCAAA (32) | | | | | | | | 2010 |
| KG809 | 333- | F- | Hex- | 55 | 5 | 0.66 | 0.64 | 0.60 | 0.01 | 4 | Gurcan and |
| | 345 | GGAAGGTGAGAG | AGGCATCAGTTC | | | | | | | | Mehlenbacher, |
| | | AAATCAAGT (33) | ATCCAA (34) | | | | | | | | 2010 |
| KG811 | 240- | F- | Ned- | 58 | 12 | 0.83 | 0.82 | 0.81 | 0.01 | 2 | Gurcan and |
| | 278 | GAACAACTGAAG | AAGGCGGCACTC | | | | | | | | Mehlenbacher, |
| | | ACAGCAAAG (35) | GCTCAC (36) | | | | | | | | 2010 |
| KG827 | 264- | Fam- | R- | 67 | 9 | 0.78 | 0.84 | 0.75 | −0.04 | 9 | Gurcan and |
| | 282 | AGAACTCCGACT | GAGGGAGCAAGT | | | | | | | | Mehlenbacher, |
| | | AATAATCCTAACC | CAAAGTTGAGAA | | | | | | | | 2010 |
| | | CTTGC (37) | GAAA (38) | | | | | | | | |
| KG830 | 279- | Ned- | R- | 67 | 9 | 0.79 | 0.78 | 0.76 | 0.00 | 9 | Gurcan and |
| | 311 | TGGAGGAAGTTTT | AAAGCAACTCAT | | | | | | | | Mehlenbacher, |
| | | GAATGGTAGTAG | AGCTGAAGTCCA | | | | | | | | 2010 |
| | | AGGA (39) | ATC (40)A | | | | | | | | |

TABLE 8
Allele sizes at 20 microsatellite markers in ‘Thompson’, its parents ‘OSU 914.082’ and ‘OSU 820.007’,
and ten other cultivars. The same microsatellite markers were used to fingerprint ‘OSU 541.147’ (US PP33,561)
in 2020 and ‘PollyO’ in 2018. ‘Thompson’ is easily distinguished from all others as shown in the table below.

| | | ‘OSU | ‘OSU | | | | | | |
|---|---|---|---|---|---|---|---|---|---|
| Marker | ‘Thompson’ | 914.082’ | 820.007’ | ‘Barcelona’ | ‘Gasaway’ | ‘McDonald’ | ‘Montebello’ | ‘PollyO’ | ‘Santiam’ |
| A613 | 151/153 | 151/169 | 153/161 | 153/161 | 161/163 | 153/169 | 151/153 | 153/167 | 151/153 |
| A614 | 158/158 | 150/158 | 132/158 | 125/132 | 143/158 | 135/158 | 125/132 | 125/158 | 132/158 |
| A616 | 152/152 | 152/152 | 152/160 | 144/152 | 150/150 | 150/160 | 152/160 | 144/152 | 150/152 |
| A640 | 362/362 | 362/374 | 362/362 | 354/374 | 362/368 | 362/368 | 362/374 | 354/354 | 362/362 |
| B617 | 285/293 | 285/293 | 285/293 | 285/289 | 291/295 | 293/295 | 285/293 | 285/295 | 285/295 |
| B619 | 158/158 | 158/166 | 158/166 | 158/172 | 172/176 | 158/172 | 160/166 | 158/166 | 158/166 |
| B634 | 222/228 | 222/228 | 228/236 | 228/228 | 222/234 | 222/228 | 232/236 | 228/236 | 222/236 |
| B657 | 218/226 | 218/226 | 218/224 | 218/222 | 224/228 | 210/218 | 218/226 | 218/218 | 218/226 |
| B671 | 239/243 | 239/239 | 243/243 | 225/229 | 237/249 | 229/237 | 225/243 | 229/249 | 225/237 |
| B709 | 229/229 | 229/229 | 229/229 | 227/235 | 229/229 | 229/229 | 229/235 | 223/227 | 229/229 |
| B733 | 173/185 | 173/181 | 181/185 | 173/175 | 175/175 | 173/175 | 175/185 | 175/181 | 175/181 |
| B741 | 181/190 | 181/190 | 181/181 | 181/190 | 190/192 | 181/192 | 181/190 | 168/188 | 190/192 |
| B749 | 216/216 | 216/216 | 216/216 | 216/216 | 214/216 | 214/216 | 216/216 | 216/216 | 216/216 |
| B751 | 144/null | 144/144 | 152/null | 144/154 | 144/144 | 144/144 | 154/154 | 144/154 | 144/144 |
| B774 | 209/220 | 209/220 | 209/209 | 209/213 | 209/215 | 209/220 | 209/213 | 209/209 | 215/220 |
| C115 | 174/194 | 174/194 | 174/194 | 174/194 | 214/218 | 174/198 | 174/198 | 194/214 | 194/198 |
| KG807 | 242/252 | 242/252 | 242/252 | 238/252 | 242/252 | 252/252 | 238/242 | 238/252 | 242/252 |
| KG809 | 339/342 | 339/342 | 339/342 | 339/339 | 339/348 | 339/339 | 339/348 | 342/342 | 339/342 |
| KG811 | 257/267 | 267/267 | 251/257 | 261/267 | 257/261 | 245/267 | 251/267 | 261/267 | 257/267 |
| KG827 | 270/270 | 270/272 | NA | 282/284 | 272/282 | 272/284 | 282/282 | 272/284 | 272/272 |

| | ‘T.G.d. | | | |
|---|---|---|---|---|
| Marker | Langhe’ | ‘T. Romana’ | ‘Wepster’ | ‘Yamhill’ |
| A613 | 153/153 | 153/177 | 159/167 | 153/163 |
| A614 | 125/135 | 125/161 | 135/158 | 132/158 |
| A616 | 150/152 | 150/152 | 152/160 | 150/150 |
| A640 | 368/368 | 372/372 | 368/374 | 354/368 |
| B617 | 285/295 | 285/291 | 293/295 | 289/295 |
| B619 | 150/166 | 158/178 | 166/172 | 158/172 |
| B634 | 228/228 | 222/232 | 228/228 | 236/236 |
| B657 | 218/226 | 218/224 | 226/226 | 218/228 |
| B671 | 239/243 | 229/239 | 239/249 | 225/243 |
| B709 | 229/229 | 227/227 | 229/235 | 229/229 |
| B733 | 173/175 | 175/179 | 173/175 | 181/185 |
| B741 | 181/188 | 168/199 | 181/190 | 181/190 |
| B749 | 216/216 | 216/216 | 214/216 | 216/216 |
| B751 | 150/154 | 144/154 | 144/144 | 152/152 |
| B774 | 209/217 | 209/215 | 209/213 | 209/217 |
| C115 | 174/174 | 174/198 | 194/194 | 198/214 |
| KG807 | 238/252 | 238/242 | 252/252 | 230/252 |
| KG809 | 339/342 | 339/342 | 342/342 | 348/348 |
| KG811 | 257/267 | 267/267 | 257/257 | 251/261 |
| KG827 | 270/270 | 268/284 | 270/282 | 282/282 |

I claim:

1. A new and distinct variety of Corylus avellana plant, substantially as illustrated and described herein.


SEQUENCE LISTING (40 sequences)

SEQ ID NO: 1 - DNA, 20 bp, synthetic construct
  source 1..20: mol_type = other DNA
cacacgccttgtcactcttt

SEQ ID NO: 2 - DNA, 20 bp, synthetic construct
  source 1..20: mol_type = other DNA
cccctttcacatgtttgctt

SEQ ID NO: 3 - DNA, 20 bp, synthetic construct
  source 1..20: mol_type = other DNA
tggcagagctttgtcagctt

SEQ ID NO: 4 - DNA, 20 bp, synthetic construct
  source 1..20: mol_type = other DNA
gcagtggaggattgctgact

SEQ ID NO: 5 - DNA, 20 bp, synthetic construct
  source 1..20: mol_type = other DNA
cactcataccgcaaactcca

SEQ ID NO: 6 - DNA, 20 bp, synthetic construct
  source 1..20: mol_type = other DNA
atggcttttgcttcgttttg

SEQ ID NO: 7 - DNA, 29 bp, synthetic construct
  source 1..29: mol_type = other DNA
tgcctctgcagttagtcatcaaatgtagg

SEQ ID NO: 8 - DNA, 25 bp, synthetic construct
  source 1..25: mol_type = other DNA
cgccatataattgggatgcttgttg

SEQ ID NO: 9 - DNA, 20 bp, synthetic construct
  source 1..20: mol_type = other DNA
tccgtgttgagtatggacga

SEQ ID NO: 10 - DNA, 19 bp, synthetic construct
  source 1..19: mol_type = other DNA
tgtttttggtggagcgatg

SEQ ID NO: 11 - DNA, 19 bp, synthetic construct
  source 1..19: mol_type = other DNA
agtcggctccccttttctc

SEQ ID NO: 12 - DNA, 20 bp, synthetic construct
  source 1..20: mol_type = other DNA
gcgatctgacctcatttttg

SEQ ID NO: 13 - DNA, 20 bp, synthetic construct
  source 1..20: mol_type = other DNA
cctgcatccaggactcatta

SEQ ID NO: 14 - DNA, 20 bp, synthetic construct
  source 1..20: mol_type = other DNA
gtgcagaggttgcactcaaa

SEQ ID NO: 15 - DNA, 20 bp, synthetic construct
  source 1..20: mol_type = other DNA
gagagtgcgtcttcctctgg

SEQ ID NO: 16 - DNA, 19 bp, synthetic construct
  source 1..19: mol_type = other DNA
agcctcacctccaacgaac

SEQ ID NO: 17 - DNA, 21 bp, synthetic construct
  source 1..21: mol_type = other DNA
ttgccagtgcatactctgatg

SEQ ID NO: 18 - DNA, 20 bp, synthetic construct
  source 1..20: mol_type = other DNA
accagctctgggcttaacac

SEQ ID NO: 19 - DNA, 20 bp, synthetic construct
  source 1..20: mol_type = other DNA
ccaagcacgaatgaactcaa

SEQ ID NO: 20 - DNA, 20 bp, synthetic construct
  source 1..20: mol_type = other DNA
gcgggttctcgttgtacact

SEQ ID NO: 21 - DNA, 20 bp, synthetic construct
  source 1..20: mol_type = other DNA
caccctcttcaccacctcat

SEQ ID NO: 22 - DNA, 20 bp, synthetic construct
  source 1..20: mol_type = other DNA
catcccctgttggagttttc

SEQ ID NO: 23 - DNA, 20 bp, synthetic construct
  source 1..20: mol_type = other DNA
ggctgacaacacagcagaaa

SEQ ID NO: 24 - DNA, 20 bp, synthetic construct
  source 1..20: mol_type = other DNA
tcggctagggttagggtttt

SEQ ID NO: 25 - DNA, 20 bp, synthetic construct
  source 1..20: mol_type = other DNA
agctggttcttcgacattcc

SEQ ID NO: 26 - DNA, 23 bp, synthetic construct
  source 1..23: mol_type = other DNA
aaactcaaataaaacccctgctc

SEQ ID NO: 27 - DNA, 20 bp, synthetic construct
  source 1..20: mol_type = other DNA
gttttgcgagctcattgtca

SEQ ID NO: 28 - DNA, 21 bp, synthetic construct
  source 1..21: mol_type = other DNA
tgtgtgtggtctgtaggcact

SEQ ID NO: 29 - DNA, 22 bp, synthetic construct
  source 1..22: mol_type = other DNA
cattttccgcagataatacagg

SEQ ID NO: 30 - DNA, 25 bp, synthetic construct
  source 1..25: mol_type = other DNA
gtttccagatctgcctccatataat

SEQ ID NO: 31 - DNA, 18 bp, synthetic construct
  source 1..18: mol_type = other DNA
aagcaagaaagggatggt

SEQ ID NO: 32 - DNA, 21 bp, synthetic construct
  source 1..21: mol_type = other DNA
cttacagataaatggctcaaa

SEQ ID NO: 33 - DNA, 21 bp, synthetic construct
  source 1..21: mol_type = other DNA
ggaaggtgagagaaatcaagt

SEQ ID NO: 34 - DNA, 18 bp, synthetic construct
  source 1..18: mol_type = other DNA
aggcatcagttcatccaa

SEQ ID NO: 35 - DNA, 21 bp, synthetic construct
  source 1..21: mol_type = other DNA
gaacaactgaagacagcaaag

SEQ ID NO: 36 - DNA, 18 bp, synthetic construct
  source 1..18: mol_type = other DNA
aaggcggcactcgctcac

SEQ ID NO: 37 - DNA, 30 bp, synthetic construct
  source 1..30: mol_type = other DNA
agaactccgactaataatcctaacccttgc

SEQ ID NO: 38 - DNA, 28 bp, synthetic construct
  source 1..28: mol_type = other DNA
gagggagcaagtcaaagttgagaagaaa

SEQ ID NO: 39 - DNA, 29 bp, synthetic construct
  source 1..29: mol_type = other DNA
tggaggaagttttgaatggtagtagagga

SEQ ID NO: 40 - DNA, 27 bp, synthetic construct
  source 1..27: mol_type = other DNA
aaagcaactcatagctgaagtccaatc